(19) United States
(12) Patent Application Publication
(10) Pub. No.: US 2017/0056461 A1
(43) Pub. Date: Mar. 2, 2017

(54) METHOD FOR PROTECTING NEURONAL CELLS COMPRISING ADMINISTERING AN EFFECTIVE AMOUNT OF A CENTIPEDA MINIMA EXTRACT

(71) Applicant: KOREA INSTITUTE OF SCIENCE AND TECHNOLOGY, Seoul (KR)

(72) Inventors: Jae Wook LEE, Gangneung-si (KR); Ju Young LEE, Gangneung-si (KR); Jin Soo PARK, Gangneung-si (KR); Jin Wook CHA, Gangneung-si (KR); Hak Cheol KWON, Gangneung-si (KR); Su-Nam KIM, Gangneung-si (KR); Byung Hun UM, Gangneung-si (KR)

(73) Assignee: KOREA INSTITUTE OF SCIENCE AND TECHNOLOGY, Seoul (KR)

(21) Appl. No.: 15/242,762

(22) Filed: Aug. 22, 2016

(30) Foreign Application Priority Data
Aug. 24, 2015 (KR) 10-2015-0118882

(51) Int. Cl. A61K 36/28; A23L 33/105

(52) U.S. Cl. CPC A61K 36/28; A23L 33/105; A23V 2002/00

(57) ABSTRACT

The present disclosure relates to a composition which comprises Centipede minima as an active ingredient. The composition of the present disclosure exhibits an effect of protecting neuronal cells from oxidative stress and inhibiting apoptosis of neuronal cells. In addition, the composition can increase expression of antioxidant enzymes which inhibit cellular apoptosis caused by increased oxidative stress in cells. Accordingly, the composition of the present disclosure can exhibit an effect of preventing, improving or treating secondary diseases that may be caused by apoptosis of neuronal cells due to oxidative stress and can be used as a pharmaceutical or food composition.

### CROSS-REFERENCE TO RELATED APPLICATION

This application claims the priority of Korean Patent Application No. 10-2015-0118882, filed on Aug. 24, 2015, and all the benefits accruing therefrom under 35 U.S.C. §119, the contents of which in its entirety are herein incorporated by reference.

### BACKGROUND

1. Field

The present disclosure relates to a composition which comprises a Centipede minima extract.

2. Description of the Related Art

Oxidative stress to neuronal cells causes many neurological diseases. In particular, it is known to be related with palsy, amyotrophic lateral sclerosis (Lou Gehrig's disease), Parkinson's disease and Alzheimer's disease and glutamate toxicity is used as a model for inducing acute and chronic toxicity in neuronal cells (Andersen, J. K. et al. Nat. Rev. Neurosci. 2004, 5, S18-S25, Coyle, J. et al. Science 1993, 262, 689-695). Therefore, treatment of neuronal cells with high levels of glutamate leads to suppressed uptake of cysteine, decrease in glutathione and increase in reactive oxygen species.

### SUMMARY

The present disclosure is directed to providing a composition which exhibits an effect of protecting neuronal cells from oxidative stress.

In an aspect, the present disclosure provides a composition which comprises a Centipede minima extract as an active ingredient.

The composition according to an aspect of the present disclosure exhibits an effect of protecting neuronal cells from oxidative stress, inhibiting apoptosis of neuronal cells and increasing expression of antioxidant enzymes which inhibit cellular apoptosis caused by increased oxidative stress in cells. Accordingly, the composition according to an aspect of the present disclosure can exhibit an effect of preventing, improving or treating secondary diseases that may be caused by apoptosis of neuronal cells due to oxidative stress and can be used as a pharmaceutical or food composition.

### BRIEF DESCRIPTION OF THE DRAWINGS

FIG. 1 shows the cell viability of neuronal cells treated with a methanol extract of Vietnamese Centipede minima.

FIG. 2 shows the cell viability of neuronal cells treated with an ethyl acetate fraction of a methanol extract of Vietnamese Centipede minima.

FIG. 3 shows the cell viability of neuronal cells treated with a water fraction of a methanol extract of Vietnamese Centipede minima.

FIG. 4 shows the images of neuronal cells treated with a Vietnamese Centipeda minima extract (scale bar=100 μm).

FIG. 5 shows the cell viability of neuronal cells treated with a methanol extract of Korean Centipeda minima.

FIG. 6 shows the cell viability of neuronal cells treated with an ethyl acetate fraction of a methanol extract of Korean Centipeda minima.

FIG. 7 shows the electrophoresis analysis of effect of a Centipeda minima methanol extract on the AIF (apoptosis inducing factor) expression.

FIG. 8 shows the electrophoresis analysis of effect of a Centipeda minima methanol extract on the P-P38.

FIG. 9 shows the electrophoresis analysis of effect of a Centipeda minima methanol extract on the P-ERK.

FIG. 10 shows the graph of effect of a Centipeda minima methanol extract on the AIF expression.

FIG. 11 shows the graph of effect of a Centipeda minima methanol extract on the P-P38.

FIG. 12 shows the graph of effect of a Centipeda minima methanol extract on the P-ERK.

### DETAILED DESCRIPTION

In an aspect, the present disclosure may relate to a composition which comprises a Centipeda minima extract as an active ingredient.

In an aspect of the present disclosure, the Centipeda minima may be obtained in Vietnam or Korea.

In an aspect of the present disclosure, the composition may be a composition for protecting neuronal cells from oxidative stress or inhibiting apoptosis of neuronal cells.

In an aspect of the present disclosure, the oxidative stress may be induced by glutamate.

In an aspect of the present disclosure, the composition may be a composition for treating, preventing or improving a disease caused by apoptosis of neuronal cells.

In an aspect of the present disclosure, the disease caused by apoptosis of neuronal cells may be one or more selected from a group consisting of palsy, amyotrophic lateral sclerosis (Lou Gehrig's disease), Parkinson's disease, ischemic stroke, Huntington's disease and Alzheimer's disease.

In an aspect of the present disclosure, the composition may be a composition for treating, preventing or improving a neurodegenerative disease.

In an aspect of the present disclosure, the neurodegenerative disease may be one or more selected from a group consisting of palsy, amyotrophic lateral sclerosis (Lou Gehrig's disease), Parkinson's disease, ischemic stroke, Huntington's disease and Alzheimer's disease.

In an aspect of the present disclosure, the composition may be a composition for treating, preventing or improving a neurological disease caused by oxidative stress. The neurological disease caused by oxidative stress may be one or more selected from a group consisting of palsy, amyotrophic lateral sclerosis (Lou Gehrig's disease), Parkinson's disease and Alzheimer's disease.

In an aspect of the present disclosure, the composition may be a composition for promoting expression of antioxidant enzymes in neuronal cells. In an aspect of the present disclosure, the antioxidant enzyme may inhibit cellular apoptosis caused by increased oxidative stress.

In an aspect of the present disclosure, the composition may be a composition for reducing or suppressing reactive oxygen species. In an aspect of the present disclosure, the composition may be a composition for reducing or suppressing reactive oxygen species generated in cells, specifically neuronal cells.

In an aspect of the present disclosure, the composition may be a pharmaceutical or food composition.

In an aspect of the present disclosure, the Centipeda minima extract may be an extract of one or more selected from a group consisting of water, an organic solvent and a mixture thereof. Specifically, in an aspect of the present disclosure, the organic solvent may be one or more selected from a group consisting of a C₁-C₆ lower alcohol, butylene glycol and propylene glycol. More specifically, the lower alcohol may be methanol.

In an aspect of the present disclosure, the Centipeda minima extract may be a fraction fractionated from an organic solvent extract of Centipeda minima with one or more selected from a group consisting of water, an organic solvent and a mixture thereof. Specifically, in an aspect of the present disclosure, the Centipeda minima extract may be a fraction fractionated from a methanol extract of Centipeda minima with one or more selected from a group consisting of water, ethyl acetate and a mixture thereof.

In an aspect of the present disclosure, the Centipeda minima may be one or more selected from a group consisting of a leaf, flower, stem, fruit, root, a combination thereof and a whole plant of Centipeda minima. Specifically, the Centipeda minima may be the whole plant of Centipeda minima.

In an aspect of the present disclosure, the concentration of the Centipede minima extract may be 0.1-1000 μg/mL based on the total volume of the composition. Specifically, in an aspect of the present disclosure, the concentration of the Centipede minima extract may be 0.1 μg/mL or higher, 0.5 μg/mL or higher, 1 μg/mL or higher, 2 μg/mL or higher, 3 μg/mL or higher, 4 μg/mL or higher, 5 μg/mL or higher, 10 μg/mL or higher, 15 μg/mL or higher, 16 μg/mL or higher, 16.5 μg/mL or higher, 16.8 μg/mL or higher, 17.0 μg/mL or higher, 17.1 μg/mL or higher, 17.2 μg/mL or higher, 17.5 μg/mL or higher, 18 μg/mL or higher, 20 μg/mL or higher, 50 μg/mL or higher, 100 μg/mL or higher, 500 μg/mL or higher or 1000 μg/mL or higher and may be 2000 μg/mL or lower, 1000 μg/mL or lower, 500 μg/mL or lower, 100 μg/mL or lower, 50 μg/mL or lower, 20 μg/mL or lower, 18 μg/mL or lower, 17.5 μg/mL or lower, 17.2 μg/mL or lower, 17.1 μg/mL or lower, 17.0 μg/mL or lower, 16.8 μg/mL or lower, 16.5 μg/mL or lower, 16 μg/mL or lower, 15 μg/mL or lower, 10 μg/mL or lower, 5 μg/mL or lower, 4 μg/mL or lower, 3 μg/mL or lower, 2 μg/mL or lower, 1 μg/mL or lower, 0.5 μg/mL or lower or 0.1 μg/mL or lower based on the total volume of the composition, although not being limited thereto.

In the present disclosure, the “Centipede minima” refers to a plant in the genus Centipede, family Asteraceae, order Asterales, class Magnoliopsida, phylum Magnoliophyta. It has been traditionally used as folk medicine in China for rhinitis, sinusitis, relieving pain and reducing swelling and is known to have anticancer and liver-protecting effects. However, nothing is known about its neuronal cell-protecting effect.

In the present disclosure, the “extract” includes any substance extracted from a natural product, regardless of extraction method, extraction solvent, extracted ingredients or type of the extract. The term is used in a broad concept, including the substance that may be obtained by otherwise processing or treating the obtained extract. Specifically, the processing or treatment may be fermentation or enzymatic treatment of the extract. Accordingly, in the present disclosure, the extract includes a fermentation product, a concentration product and a drying product. Specifically, the extract in the present disclosure may be a fermentation product.

In an aspect of the present disclosure, the “Centipeda minima extract” includes any substance extracted from, a Centipeda minima, regardless of extraction method, extraction solvent, extracted ingredients or type of the extract. It includes the substance that may be extracted by treating with heat, an acid, a base, an enzyme, etc. and the term is used in a broad concept, including the substance that may be obtained by otherwise processing or treating the obtained extract. Specifically, the processing or treatment may be fermentation or enzymatic treatment of the Centipeda minima extract. Accordingly, in the present disclosure, the Centipeda minima extract may be a fermentation product.

In an aspect of the present disclosure, the “Centipeda minima” may be an extract, live Centipeda minima, a pulverization product of live Centipeda minima, a drying product of live Centipeda minima, a dried pulverization product of live Centipeda minima or a fermentation product of Centipeda minima, although not being limited thereto. The Centipeda minima used in the present disclosure is not limited as to how it is obtained. It may be either cultivated or purchased commercially and the aerial or root part of the plant may be used wholly or partly. More specifically, one or more selected from a group consisting of the leaf, flower, stem, fruit, root, a combination thereof and the whole plant of Centipeda minima may be used. In the present disclosure, the Centipeda minima needs not necessarily to be dried and is not particularly limited as long as it is suitable to extract the active ingredients of Centipeda minima.

In an aspect of the present disclosure, the water may be distilled water or purified water and the organic solvent may be one or more selected from a group consisting of an alcohol, e.g., a C₁-C₅ lower alcohol, acetone, ether, ethyl acetate, diethyl ether, ethyl methyl ketone and chloroform, although not being limited thereto.

In an aspect of the present disclosure, the Centipeda minima extract may be a C₁-C₆ alcohol extract of Centipeda minima. Specifically, the alcohol may be methanol or ethanol.

In an aspect of the present disclosure, the Centipeda minima extract may be obtained by a preparation method comprising a step of extracting Centipeda minima with water, an organic solvent or a mixture thereof.

In an aspect of the present disclosure, the Centipeda minima extract may be a crude extract of a solvent selected from a group consisting of water, an organic solvent and a combination thereof. The organic solvent may be a C₁-C₆ alcohol. Specifically, the C₁-C₆ alcohol may be methanol or ethanol. In an aspect of the present disclosure, when extracting Centipeda minima with a solvent, about 5-15 times (v/w), specifically about 10 times, of the solvent may be added to the Centipeda minima, although not being limited thereto.

In an aspect of the present disclosure, the extraction may be performed by hot water extraction, ethanol extraction, heating extraction, cold extraction, reflux extraction, reflux condensation extraction, ultrasonic extraction, etc. However, any extraction method obvious to those skilled in the art can be employed without limitation. Specifically, the extraction may be performed by hot water extraction or ethanol extraction.

In an aspect of the present disclosure, although the extraction may also be performed at room temperature, it may be performed at elevated temperatures, specifically at about 40-100° C., more specifically at about 65-75° C., although not being limited thereto. Extraction time may be about 2-48 hours, specifically 18-36 hours, more specifically 20-28 hours, most specifically 22-26 hours. However, the extraction time may vary depending on conditions such as extraction solvent, extraction temperature, etc. without being limited thereto. The extraction may be performed more than once in order to obtain the active ingredients in larger quantities. The extraction may be performed specifically 1-5 times, more specifically 3 times.

In an aspect of the present disclosure, the Centipede minima extract may include a crude extract of Centipede minima as described above and may include a soluble fraction obtained by further extracting the crude extract with an organic solvent of low polarity. In an aspect of the present disclosure, the organic solvent may be hexane, methylene chloride, ethyl acetate, n-butanol, etc., although not being limited thereto. The extract or the soluble fraction of the extract may be used either as it is or after it is filtered and concentrated. Also, it may be used after it is filtered and concentrated and then dried.

In an aspect of the present disclosure, the drying may be performed by evaporation drying, spray drying or freeze-drying. Specifically, freeze-drying may be performed at −50 to −70° C. for 3-4 days.

The pharmaceutical composition according to an aspect of the present disclosure may be prepared into a formulation for oral or parenteral administration using a commonly used diluent or excipient such as a filler, an extender, a binder, a wetting agent, a disintegrant, a surfactant, etc. Solid formulations for oral administration include a tablet, a pill, a powder, a granule, a soft or hard capsule, etc. The solid formulation is prepared by adding one or more excipient, e.g., starch, calcium carbonate, sucrose, lactose, gelatin, etc., to the active ingredient. In addition to a simple excipient, a lubricant such as magnesium stearate, talc, etc. is also used. Liquid formulations for oral administration include a suspension, a liquid formulation for internal use, an emulsion, a syrup. They may comprise, in addition to a commonly used simple diluent such as water or liquid paraffin, various other excipients such as a wetting agent, a sweetener, an aromatic, a preservative, etc. Formulations for parenteral administration include a sterilized aqueous solution, a non-aqueous solution, a suspension, an emulsion, a freeze-dried formulation and a suppository. For the non-aqueous solution or suspension, propylene glycol, polyethylene glycol, a vegetable oil such as olive oil, an injectable ester such as ethyl oleate, etc. may be used as a solvent. As a base of the suppository, witepsol, macrogol, Tween 61, cocoa butter, laurin butter, glycerogelatin, etc. may be used.

The active ingredient of the composition according to an aspect of the present disclosure may be pharmaceutically administered either as it is or as a pharmaceutically acceptable salt thereof. In addition, it may be used either alone or in suitable combination with other pharmaceutically active compounds. The salt is not particularly limited as long as it is pharmaceutically acceptable. For example, a hydrochloride, a sulfate, a nitrate, a phosphate, a hydrofuloride, a hydrobromide, a formate, an acetate, a tartrate, a lactate, a citrate, a fumarate, a maleate, a succinate, a methanesulfonate, a benzenesulfonate, a toluenesulfonate, a naphthalenesulfonate, etc. may be used.

The composition according to an aspect of the present disclosure may be administered parenterally or orally depending on purposes. A daily dosage of 0.1-500 mg, specifically 1-100 mg, per kg body weight may be administered once or several times a day. The administration dosage for a particular patient may vary depending on the body weight, age, sex and physical condition of the patient, diet, administration time, administration method, excretion rate, severity of a disease, etc.

The pharmaceutical composition according to an aspect of the present disclosure may be prepared into an oral formulation such as a powder, a granule, a tablet, a soft or hard capsule, a suspension, an emulsion, a syrup, a drink, an aerosol, etc., a formulation for extremal application to skin such as an ointment, a cream, etc. or any type of pharmaceutically suitable formulation such as a suppository, an injection, a sterile solution for injection, etc. according to common methods. Specifically, it may be prepared into an injection or a formulation for extremal application to skin.

The composition according to an aspect of the present disclosure may be administered to mammals such as rat, mouse, cattle, human, etc. through various routes including parenteral and oral routes. All modes of administration may be expected. For example, it may be administered orally, transdermally, rectally, intravenously, intramuscularly, subcutaneously, intrauterinarily or intracerebrovascularly.

The composition according to an aspect of the present disclosure may be administered through various routes that may be readily selected by those skilled in the art. In particular, the pharmaceutical composition according to the present disclosure may be administered through skin as a formulation for extremal application to skin.

In an aspect of the present disclosure, the food composition may be a health functional food composition.

The formulation of the food composition according to an aspect of the present disclosure is not particularly limited. For example, it may be prepared into a tablet, a granule, a powder, a liquid such as a drink, a caramel, a gel, a bar, etc. Each formulation of the food composition may further comprise, in addition to the active ingredient, ingredients commonly used in the art depending on the particular formulation or purposes. In this case, a synergic effect may be achieved.

Determination of the administration dosage of the active ingredient of the food composition according to an aspect of the present disclosure is within the level of those skilled in the art. A daily dosage may be, for example, 0.1-5000 mg/kg/day, more specifically 50-500 mg/kg/day. However, the administration dosage may be varied depending on various factors such as the age and physical condition of a subject, presence of complication(s), etc. without being limited thereto.

The food composition according to an aspect of the present disclosure may be various foods, e.g., chewing gum, caramel, candy, ice cake, confectionery, etc., drinks such as soft drink, mineral water, alcoholic beverage, etc. or health functional foods including vitamins and minerals.

The food composition according to an aspect of the present disclosure may further comprise various nutrients, vitamins, minerals (electrolytes), flavors such as synthetic and natural flavors, colorants, extenders (cheese, chocolate, etc.), pectic acid and its salts, alginic acid and its salts, organic acids, protective colloidal thickeners, pH control agents, stabilizers, antiseptics, glycerin, alcohols, carbonating agents used in carbonated drinks, etc. In addition, the functional food composition according to an aspect of the present disclosure may comprise a pulp used to prepare natural fruit juice, fruit juice drinks or vegetable drinks. These ingredients may be used either independently or in combination. The addition amount of these additives is no great importance. Usually, they are comprised in an amount of about 0-20 parts by weight based on 100 parts by weight of the composition of the present disclosure.

Hereinafter, the present disclosure will be described in detail through examples and test examples. However, the following examples and test examples are for illustrative purposes only and it will be apparent to those of ordinary skill in the art that the scope of the present disclosure is not limited by them.

### Example 1

### Preparation of Vietnamese Centipede minima Extract

10 g of the whole plant of Vietnamese Centipede minima (including the root, stem, leaf, etc.) was treated with 300 mL of methanol and stored at room temperature for 3 days. After filtration and concentration, 100 mg of a Centipede minima extract was obtained in the form of a viscous, dark-brown oil. The Vietnamese Centipede minima extract prepared as described above was acquired from the Institute of Natural Products Chemistry of the Vietnam Academy of Science and Technology.

The obtained methanol extract of Vietnamese Centipede minima was dissolved in a solvent mixture of ethyl acetate (40 mL) and water (10 mL) and then separated into an organic layer and an aqueous layer. As a result, an ethyl acetate fraction (30 mg) and a water fraction (20 mg) of the methanol extract were obtained. Samples were prepared by dissolving the obtained methanol extract of Centipede minima, the ethyl acetate fraction and the water fraction in dimethyl sulfoxide (DMSO) at a concentration of 10 mg/mL.

### Example 2

### Preparation of Korean Centipede minima Extract

10 g of the whole plant of Centipede minima (including the root, stem, leaf, etc.) harvested in Yeongcheon, Gyeongbuk, Korea was treated with 300 mL of methanol and stored at room temperature for 3 days. After filtration and concentration, 100 mg of a Centipede minima extract was obtained in the form of a viscous, dark-brown oil.

The obtained methanol extract of Korean Centipede minima was dissolved in a solvent mixture of ethyl acetate (40 mL) and water (10 mL) and then separated into an organic layer and an aqueous layer. As a result, an ethyl acetate fraction (30 mg) and a water fraction (20 mg) of the methanol extract were obtained. Samples were prepared by dissolving the obtained methanol extract of Centipede minima, the ethyl acetate fraction and the water fraction in dimethyl sulfoxide (DMSO) at a concentration of 10 mg/mL.

### Test Example 1

### Measurement of Cell Viability of Neuronal Cells Under Oxidative Stress

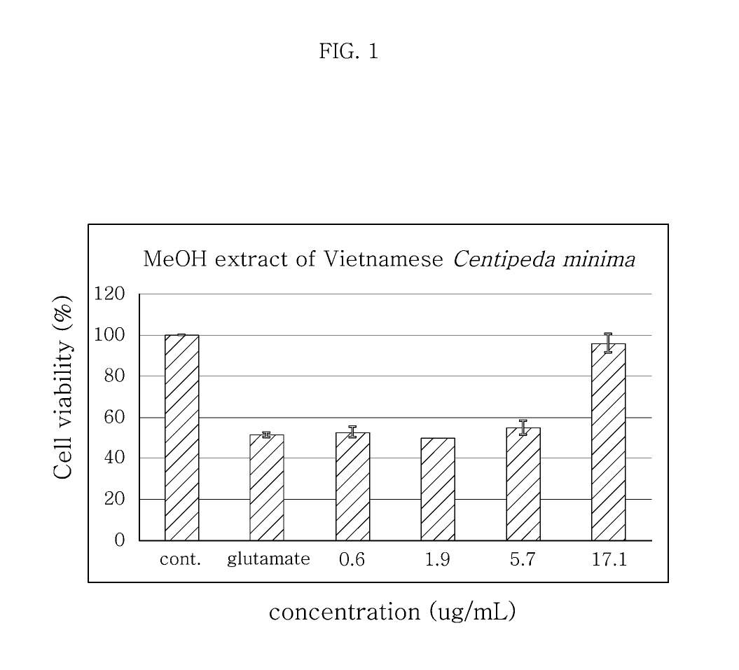

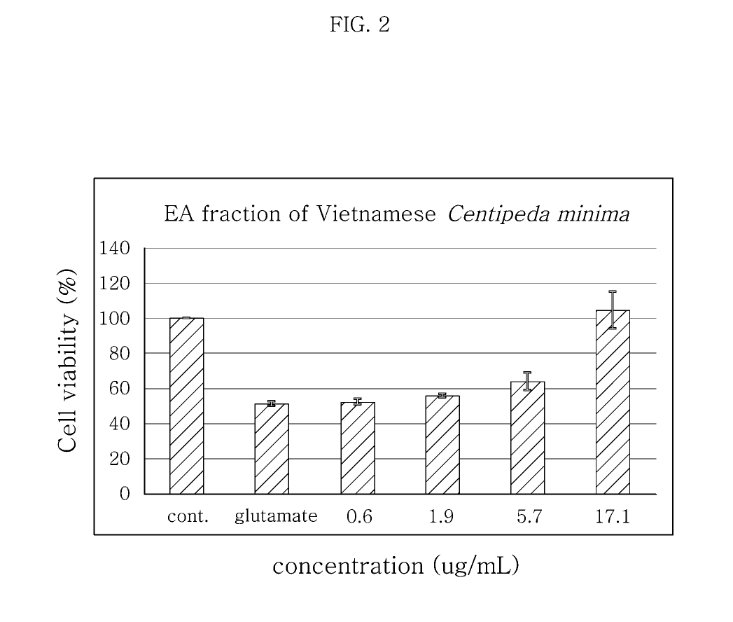

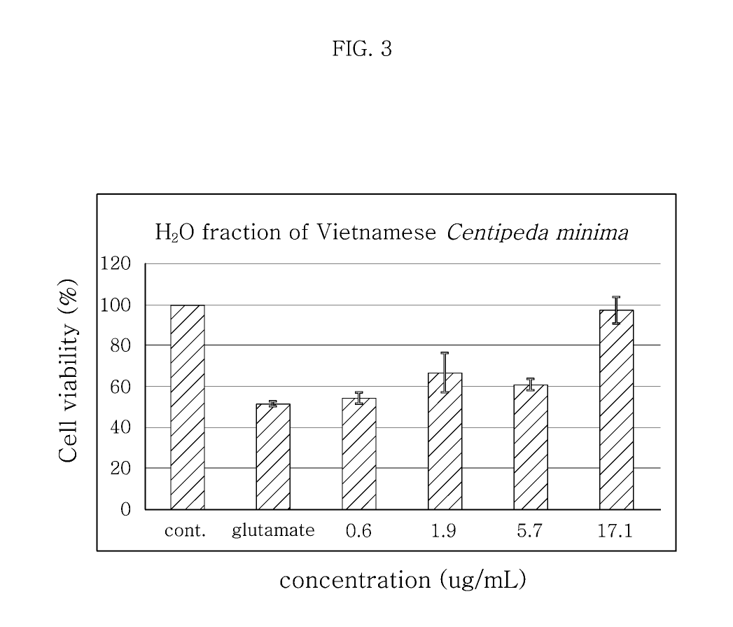

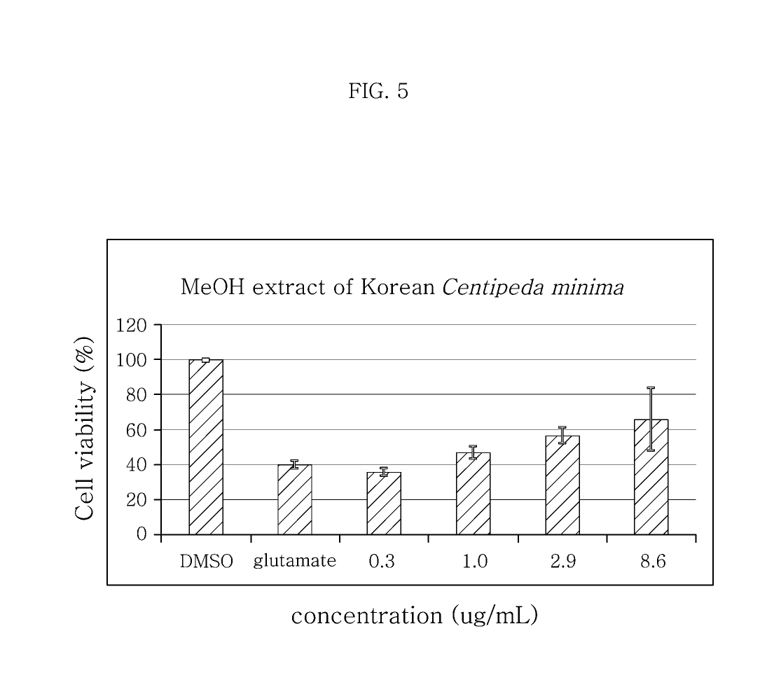

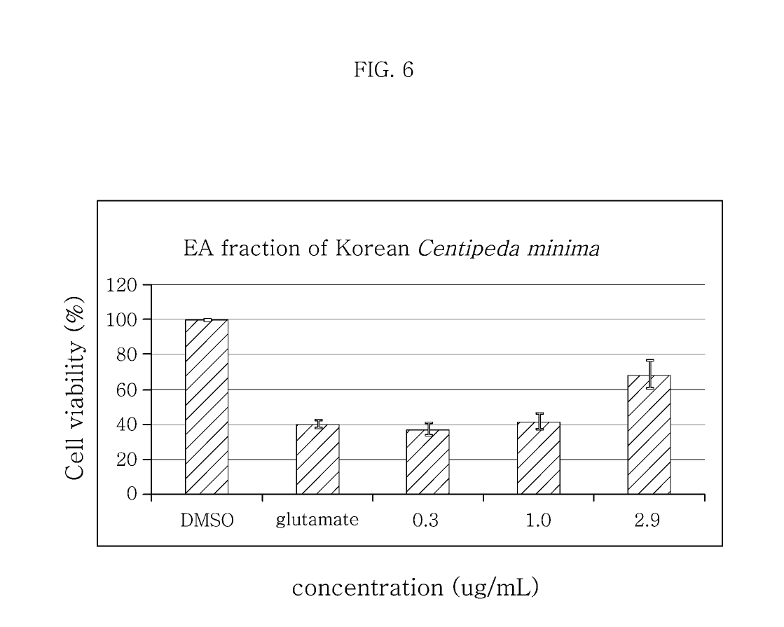

HT-22 cells derived from mouse hippocampal neurons (Salk Institute, USA) were subcultured in DMEM (Life Technologies, 11965-092) supplemented with 10% fetal bovine serum (GIBCO) and 1% penicillin-streptomycin based on the total volume of the medium while replacing the medium once in 3 days. After adding 100 μL of the medium to each well of a 96-well plate, 5,000 HT-22 cells were added to each well. The cells were cultured in a 5% CO₂ incubator at 37° C. for 24 hours and then treated with the samples (10 mg/ml) of Example 1 and Example 2 (the methanol extract, the ethyl acetate fraction and the water fraction of Examples 1 and 2) dissolved in DMSO at the concentrations described in FIGS. 1-3 and FIGS. 5-6 (Example 1: 0.6 μg/mL, 1.9 μg/mL, 5.7 μg/mL, 17.1 μg/mL; Example 2: 0.3 μg/mL, 1.0 μg/mL, 2.9 μg/mL, 8.6 μg/mL). 10 mM glutamate was added 2 hours later and cell viability was measured after culturing for 24 hours. For this, absorbance was measured at 450 nm using the EZ-Tox reagent (Daeil Lab Service) and a plate reader. A medium treated with nothing and a medium treated with 10 mM glutamate only were used as control groups. The result is shown in FIGS. 1-3 and FIGS. 5-6.

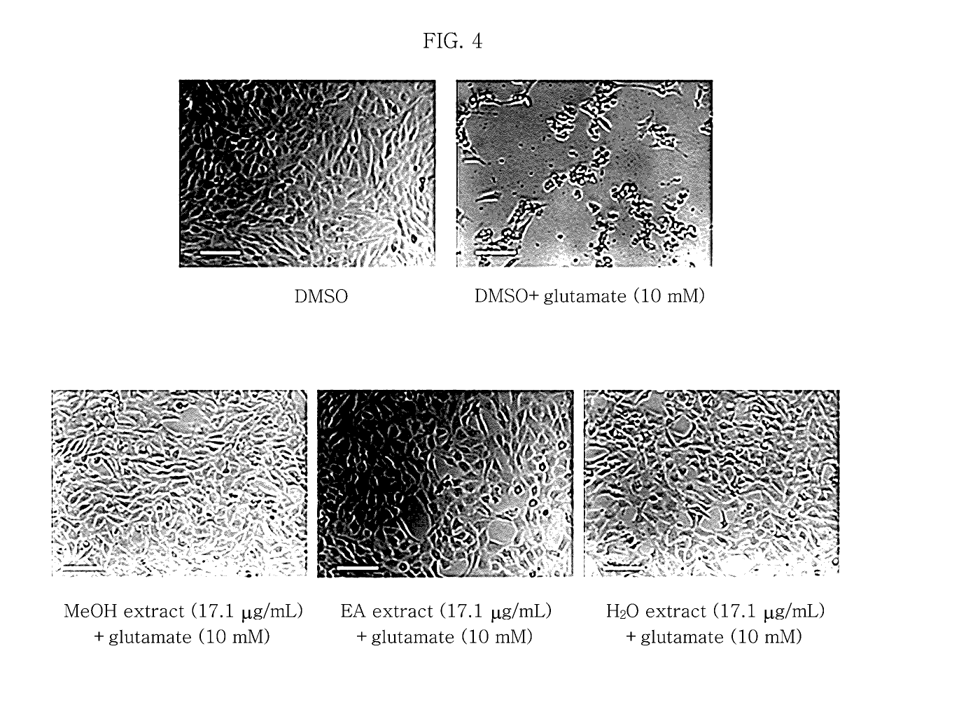

HT-22 cells cultured in the same manner as described above were treated with the Vietnamese Centipede minima extract of Example 1 (the methanol extract, the ethyl acetate fraction and the water fraction) at 17.1 μg/mL. After adding 10 mM glutamate 2 hours later and culturing the cells in a 5% CO₂ incubator at 37° C. for 24 hours, cell viability was evaluated by imaging the cultures with Operetta (high content imaging system, Perkin Elmer) using a 10× lens. A medium treated with nothing and a medium treated with 10 mM glutamate only were used as control groups. The result is shown in FIG. 4 (scale bar=100 μm).

From FIGS. 1-3 and FIGS. 5-6, it can be seen that the Vietnamese or Korean Centipede minima extract according to an aspect of the present disclosure is effective in increasing the survivability of neuronal cells apoptosis of which is induced by glutamate.

From FIG. 4, it can be seen that apoptosis of neurons is induced by glutamate. However, when the cells are treated with the methanol extract of Vietnamese Centipede minima or the ethyl acetate fraction or the water fraction of the methanol extract according to an aspect of the present disclosure and glutamate at the same time, the cell viability was similar to that of the control group treated only with DMSO.

Accordingly, from FIGS. 1-6, it can be seen that the Centipede minima extract according to an aspect of the present disclosure exhibits an effect of protecting neuronal cells from oxidative stress and inhibiting apoptosis of neuronal cells and thus can treat, prevent or improve neurological diseases induced by apoptosis of neuronal cells or caused by oxidative stress. Also, the Centipede minima extract according to an aspect of the present disclosure can promote the expression of antioxidant enzymes which inhibit apoptosis caused by increased oxidative stress in neuronal cells.

According to the common knowledge in the field of the present disclosure (see non-patent documents 2, 3 and 4), glutamate is a major neurotransmitter in the central nervous system and causes neurotoxicity and neurodegenerative diseases when present at high concentration. Those skilled in the art have also used a glutamate-induced oxidative stress model for the study of the cause of neurodegenerative diseases by applying it to HT-22 cells. Because the Centipede minima extract according to an aspect of the present disclosure exhibits an effect of inhibiting apoptosis of neuronal cells induced by glutamate in HT-22 cells, it will be obvious to those skilled in the art that the Centipede minima extract is effective in treating, preventing or improving neurodegenerative diseases.

### Test Example 2

### Analysis of Effects on AIF (Apoptosis Inducing Factor) Expression and MAPK (Mitogen-Activated Protein Kinases)

The HT-22 cells derived from mouse hippocampal neurons were cultured in a 5% or less CO₂ incubator at 37° C. and subcultured every 2 days. The medium was a growth medium which contained DMEM (Hyclone) supplemented with 10% fetal bovine serum (GIBCO) and 1% penicillin (Life technology) based on the total volume of the medium.

The cells were cultured in 10 cm dish (Corning Incorporated, 430167). 3.5×10̂5 cells were added to each dish (3.5×10̂5 cells/dish). After 24 hours, when the confluency of cells reached 50-60%, the Korean Centipede minima methanol extract (200 μg/mL) was added. After 2 hours, glutamate (10 mM) was added, and the cells were incubated for 12 hours. After incubation, the cells were harvested as pellet form, and 200 μL of RIPA buffer (CST#9806, with Phenylmethanesulfonyl Fluoride (PMSF), Cell signaling technology, USA) was added (sprayed). After incubation on ice for 30 minutes, sonication was conducted. After that, cell lysates were centrifuged at 12,000 rpm for 10 minutes at 4° C. Supernatant fluid except for pellet was taken to obtain proteins. Contents of protein in cell lysates were quantified with protein assay kit II (Bio-Rad#5000002). After quantification, 4× Lammli sample buffer and 2-mercaptoethanol was added to the prepared samples, and the samples were heated for 5 minutes at 80° C. 10% SDS-polyacrylamide Gel samples (20 μg/well) were added (sprayed) to each well. SDS-PAGE electrophoresis was performed (120V, 90 min). After that, the transfer was performed (100V, 90 min) using a PDVF membrane. PDVF membranes were washed with TBS-T buffer for 10 min. PDVF mwmbranes were blocked with buffer 5% non-fat dry milk and 1% bovine serum albumin). AIF primary antibody (Santa Cruz SC-9416, SANTA CRUZ BIOTECHNOLOGY, INC., USA), p-ERK primary antibody (CST#9101, Cell signaling technology), p-p38 primary antibody (CST#9211), beta-actin primary antibody (CST#4967) were interacted overnight at 4° C. After membrane was washed, secondary antibodies (anti-rabbit-HRP, anti-mouse-HRP) was interacted for 1 hour at room temperature. The membranes were washed with TBS-T buffer again. In the case of p-p38 and p-ERK, GAPDH was used as loading control substance. After that, the enhanced chemiluminescence solution was added, and the expression of proteins was analyzed with LAS-1000 (Fujifilm).

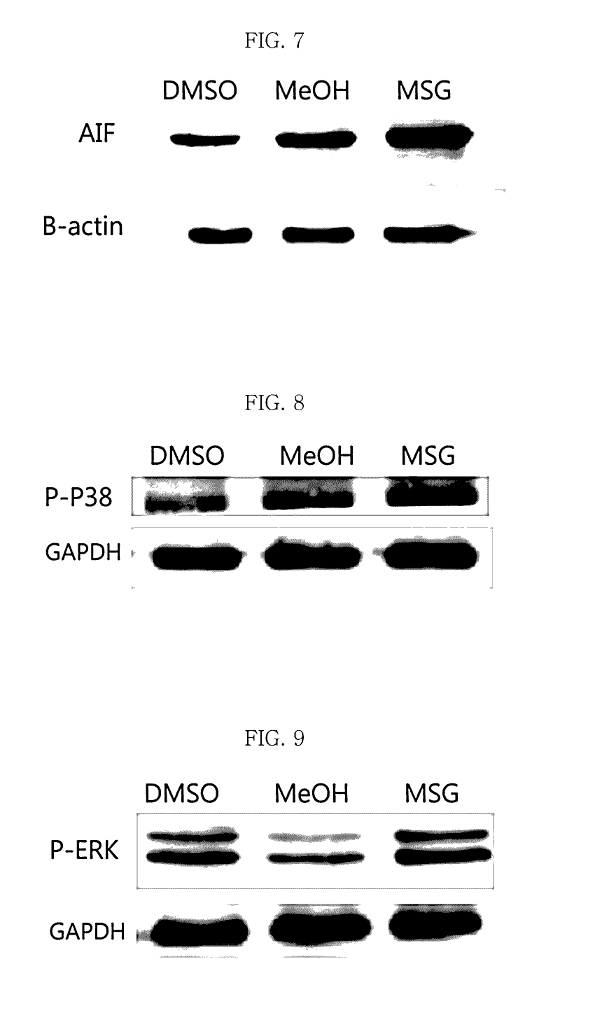

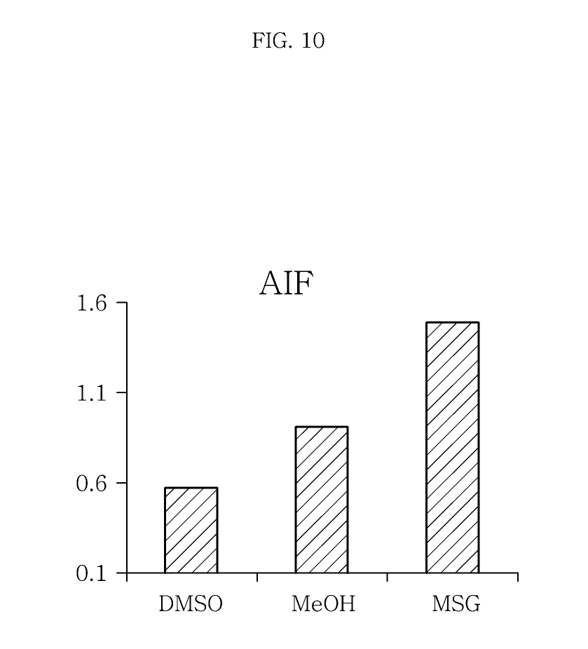

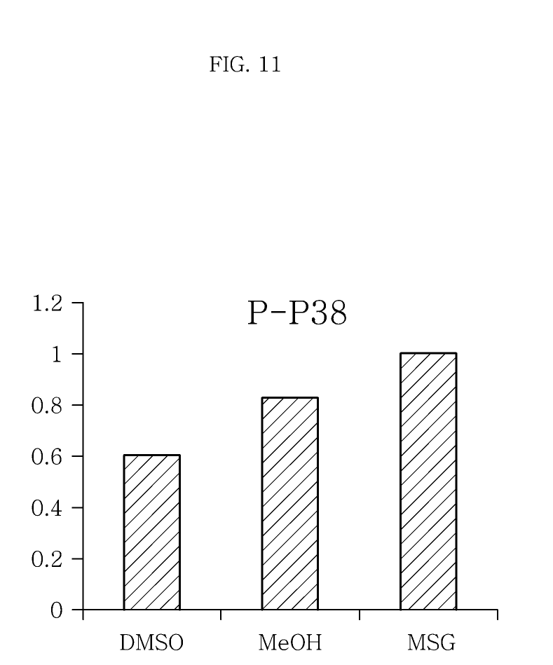

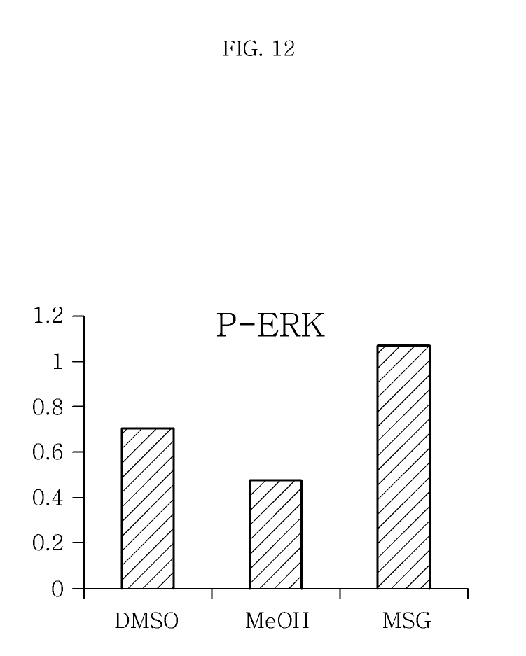

The results are shown in FIGS. 7-12. In FIGS. 7-12, “MeOH” means the sample which was added with the Centipede minima methanol extract and glutamate (10 mM). “MSG” means the samples which were added with glutamate (mono sodium glutamate, 10 mM) except for the Centipede minima methanol extract. The numbers on y-axis of FIG. 10-12 mean the relative expression amount of target protein assuming that the expression amount of the beta-actin is 1.

Glutamate added to HT-22 cells expresses AIF and activates MAPK signal, causing the apoptosis. However, the Centipede minima methanol extract remarkably inhibits expression of AIF and activation of MAPK signal. Thus, the Centipede minima extract inhibits the apoptosis caused by glutamate (FIGS. 7-12).

Hereinafter, the present disclosure will be described in detail through formulation examples. However, the following formulation examples are for illustrative purposes only and it will be apparent to those of ordinary skill in the art that the scope of the present disclosure is not limited by them.

### Formulation Example 1

### Soft Capsule

A soft capsule filling solution was prepared by mixing 8 mg of the methanol extract of Centipeda minima of Example 1, 9 mg of vitamin E, 9 mg of vitamin C, 2 mg of palm oil, 8 mg of hydrogenated vegetable oil, 4 mg of yellow beeswax and 9 mg of lecithin according to a common method. A soft capsule was prepared by filling 400 mg of the solution per capsule. Separately from this, a soft capsule sheet was prepared from 66 parts by weight of gelatin, 24 parts by weight of glycerin and 10 parts by weight of sorbitol solution and a soft capsule comprising 400 mg of the composition according to the present disclosure was prepared by filling the filling solution therein.

### Formulation Example 2

### Tablet

8 mg of the methanol extract of Centipeda minima of Example 1, 9 mg of vitamin E, 9 mg of vitamin C, 200 mg of galactooligosaccharide, 60 mg of lactose and 140 mg of maltose were mixed. After granulating using a fluidized-bed drier, 6 mg of sugar ester was added. Then, a tablet was prepared by tableting 500 mg of the resulting composition according to a common method.

### Formulation Example 3

### Drink

8 mg of the methanol extract of Centipeda minima of Example 1, 9 mg of vitamin E, 9 mg of vitamin C, 10 g of glucose, 0.6 g of citric acid and 25 g of oligosaccharide syrup were mixed. After adding 300 mL of purified water, 200 mL of the resulting solution was filled per bottle. Then, a drink was prepared by sterilizing at 130° C. for 4-5 seconds.

### Formulation Example 4

### Granule

8 mg of the methanol extract of Centipeda minima of Example 1, 9 mg of vitamin E, 9 mg of vitamin C, 250 mg anhydrous crystalline glucose and 550 mg of starch were mixed and granulated using a fluidized-bed granulator. The resulting granule was filled in a pouch.

### Formulation Example 5

### Injection

An injection was prepared according to a common method with the composition described in Table 1.

TABLE 1

| Ingredients | Contents |
|---|---|
| Methanol extract of Centipeda minima of Example 1 | 10-50 mg |
| Sterilized distilled water for injection | adequate |
| pH control agent | adequate |

### Formulation Example 6

### Health Functional Food

A health functional food was prepared according to a common method with the composition described in Table 2.

TABLE 2

| Ingredients | Contents | |
|---|---|---|
| Methanol extract of Centipeda minima of Example 1 | 20 | mg |
| Vitamin A acetate | 70 | μg |
| Vitamin E | 1.0 | mg |
| Vitamin B₁ | 0.13 | mg |
| Vitamin B₂ | 0.15 | mg |
| Vitamin B₆ | 0.5 | mg |
| Vitamin B₁₂ | 0.2 | μg |
| Vitamin C | 10 | mg |
| Biotin | 10 | μg |
| Nicotinamide | 1.7 | mg |
| Folic acid | 50 | μg |
| Calcium pantothenate | 0.5 | mg |
| Ferrous sulfate | 1.75 | mg |
| Zinc oxide | 0.82 | mg |
| Magnesium carbonate | 25.3 | mg |
| Potassium phosphate monobasic | 15 | mg |
| Calcium phosphate dibasic | 55 | mg |
| Potassium citrate | 90 | mg |
| Calcium carbonate | 100 | mg |
| Magnesium chloride | 24.8 | mg |

The compositions of the vitamins and minerals described above, which are given as a specific exemplary embodiment, may be varied as desired.

### Formulation Example 7

### Health Drink

A health drink was prepared according to a common method with the composition described in Table 3.

TABLE 3

| Ingredients | Contents | |
|---|---|---|
| Methanol extract of Centipeda minima of Example 1 | 1000 | mg |
| Citric acid | 1000 | mg |
| Oligosaccharide | 100 | g |
| Taurine | 1 | g |

| | |
|---|---|
| Purified water | Balance |

The above ingredients were mixed according to a common health drink preparation method. After heating at 85° C. for about 1 hour under stirring, the resulting solution was sterilized.

What is claimed is:

1. A method for protecting neuronal cells from oxidative stress comprising administering an effective amount of a Centipede minima extract to a subject in need thereof.

2. The method according to claim 1, wherein the extract inhibits apoptosis of neuronal cells.

3. The method according to claim 1, wherein the extract treats, prevents or improves a neurodegenerative disease.

4. The method according to claim 1, wherein the extract promotes expression of antioxidant enzymes in neuronal cells.

5. The method according to claim 1, wherein the extract reduces or suppresses reactive oxygen species in neuronal cells.

6. The method according to claim 1, wherein the oxidative stress is induced by glutamate.

7. The method according to claim 3, wherein the neurodegenerative disease is one or more selected from a group consisting of palsy, amyotrophic lateral sclerosis (Lou Gehrig's disease), Parkinson's disease, ischemic stroke, Huntington's disease and Alzheimer's disease.

8. The method according to claim 1, wherein the Centipeda minima is one or more selected from a group consisting of a leaf, a flower, a stem, a fruit, a root, a combination thereof and a whole plant of Centipeda minima.

9. The method according to claim 8, wherein the Centipeda minima is a whole plant of Centipeda minima.

10. The method according to claim 1, wherein the Centipeda minima extract is an extract of one or more selected from a group consisting of water, an organic solvent and a mixture thereof.

11. The method according to claim 10, wherein the organic solvent is one or more selected from a group consisting of a C₁-C₆ lower alcohol, butylene glycol and propylene glycol.

12. The method according to claim 11, wherein the lower alcohol is methanol.

13. The method according to claim 10, wherein the Centipeda minima extract is a fraction fractionated from an organic solvent extract of Centipeda minima with one or more selected from a group consisting of water, ethyl acetate and a mixture thereof.

14. The method according to claim 13, wherein the Centipeda minima extract is a fraction fractionated from a methanol extract of Centipeda minima with one or more selected from a group consisting of water, ethyl acetate and a mixture thereof.

15. The method according to claim 1, wherein the Centipeda minima extract is administered in a form of a composition and the composition comprises the Centipeda minima extract at a concentration of 0.1-1000 μg/mL in the composition based on the total volume of the composition.

16. The method according to claim 1, wherein the Centipeda minima is obtained in Vietnam or Korea.

17. The method according to claim 15, wherein the composition is a pharmaceutical or a food composition.
